(19) United States
(12) Patent Application Publication
(10) Pub. No.: US 2020/0392465 A1
(43) Pub. Date: Dec. 17, 2020

(54) A PROCESS OF PREPARING BUCCAL EPITHELIAL CELL SUSPENSION AND ITS USE

(71) Applicant: Regrow Biosciences Pvt. Ltd., Mumbai (IN)

(72) Inventors: Satyen SANGHAVI, Mumbai (IN); Vinayak KEDAGE, Lonavala (IN); Charul BHANJI, Mumbai (IN)

(73) Assignee: Regrow Biosciences Pvt. Ltd., Mumbai (IN)

(21) Appl. No.: 15/761,396

(22) PCT Filed: Nov. 10, 2017
(86) PCT No.: PCT/IN2017/000129

§ 371 Date: Mar. 19, 2018

(30) Foreign Application Priority Data
Nov. 15, 2016 (IN) 201621038900

(51) Int. Cl. C12N 5/071; C12N 5/00; A61K 9/00

(52) U.S. Cl. CPC C12N 5/0684; C12N 5/0068; C12N 2533/72; C12N 2533/30; A61K 9/0034

(57) ABSTRACT

A process of preparing buccal epithelial cell suspension and cystoscopically implanting the cell suspension in the defect site of the adult human urethra.

### BACKGROUND OF THE INVENTION

The present invention relates to a process of preparing buccal epithelial cell suspension and cystoscopically implanting the suspension in the defect site of the adult human urethra.

United States Patent publication number 2010/0184220 (assigned to M/s. The Technische University Dresden) claims a tissue transplant construct comprising a biocompatible acellular membrane and microvascular bladder endothelial cells for the reconstruction of a human or animal organ. However, the present invention comprises buccal mucosal epithelial cells devoid of any membrane or scaffold.

European Urology 53 (2008) 1263-1271 titled Tissue-Engineered Buccal Mucosa Urethroplasty—Clinical Outcomes discloses use of autologous tissue engineered buccal mucosa comprising keratinocytes and fibroblasts seeded on the papillary surface of DED (de-epidermised dermis) for treating urethral strictures. However, the present invention comprises autologous buccal epithelial cell suspension which will not be seeded on DED.

European Urology Supplement 2014:13:eV60 titled Reconstruction or extended urethral stricture with tissue engineered autologous buccal mucosal graft (Mukocell) discloses expansion and culturing a 5×5 mm oral mucosal graft on the surface of a collagen scaffold which is implanted as an onlay after three weeks. The cells are seeded on collagen membrane for propagation (passage number P1). Normally, ≥1.×10⁵ cells per cm² would be embedded on the graft. The present invention does not use any cell seeded graft, and the autologous buccal epithelial cell suspension are cultured up to 2 weeks and mixed with gel before implantation.

However, the process of the present invention has lower passage number of P0 which may not alter the morphology, growth rate, protein expression of the cells as compared to cells prepared using passage number (P1).

### OBJECT OF THE INVENTION

The object of the present invention is a process to prepare autologous buccal epithelial cell suspension to repair defects in adult urethra. The process is carried out using small amount of buccal mucosal tissue.

Another object iscystoscopically implanting the autologous buccal epithelial cell suspension into the defect site of the adult human urethra with or without a biocompatible delivery system such as combination of fibrinogen and/or thrombin and/or thermo-reversible gelation polymer (TGP) gel and/or collagen and/or chitosan and the like.

### SUMMARY OF THE INVENTION

A process of preparing autologous buccal epithelial cell suspension comprising
(a) harvesting buccal mucosal tissue from subject;
(b) treating the tissue from (a) with chemical dissociation agent;
(c) washing the tissue sample from (b) with nutrient medium;
(d) separating the epidermis from dermis of the washed tissue;
(e) mincing, filtering to obtain uniform epithelial cell suspension from epidermis;
(f) optionally seeding to enable cell multiplication;
(g) harvesting the cellular monolayer with enzyme(s);
(h) centrifuging, discarding the supernatant;
(i) mixing with nutrient medium;
(j) analyzing the cell suspension;
(k) filling 0.4 ml of the cell suspension in V shaped 1 ml vials; and
(l) optionally transporting to the same subject as in (a).

A method of cystoscopically implanting the autologous buccal epithelialcell suspension into the defect site of the adult human urethra comprising
(a) harvesting human buccal mucosal tissue from the inner cheek of the adult human;
(b) subjecting the tissue from (a) to chemical dissociation agent;
(c) washing the tissue sample from (b) with nutrient medium;
(d) separating the epidermis from dermis or/the washed tissue;
(e) mincing, filtering to obtain uniform epithelial cell suspension from epidermis.
(f) optionally seeding to enable cell multiplication;
(g) harvesting the cellular monolayer with enzyme(s);
(h) centrifuging, discarding the supernatant;
(i) mixing with nutrient medium;
(j) analyzing the cell suspension;
(k) optionally filling and transporting 0.4 ml of the cell suspension in V shaped 1 ml vials; and
(l) optionally mixing with gel while cystoscopically implanting the autologous buccal epithelial cell suspension into the defect site of the adult human urethra.

### DESCRIPTION OF THE INVENTION

Urethral stricture is one of the oldest known urological diseases, and continues to be a common and challenging urologic condition. A urethral stricture is scarring in or around the urethra that narrows or blocks the passageway through which urine flows from the bladder. Urethral strictures are more common in men than in women.

The treatment of urethral strictures can be divided into three main categories; non-operative, endourological procedures and open surgical reconstruction.

The currently used treatments for managing urethral stricture are as follows
1. Open surgical reconstruction procedures
2. In stricture resection and end-to-end anastomosis; and
3. In graft urethroplasty

Irrespective of the procedure used, the % recurrence of urethral stricture is high to the tune of about 40.0%-50.0%. Also, it is reported that morbid surgical procedure incurs a high rate of infection.

Another technique being used is the application of autologous buccal epithelial cells expanded & delivered through cellular or acellular scaffold.

We have surprisingly found that buccal epithelial cells from a subject may be isolated, proliferated, characterized and cystoscopically implanted into the subject without scaffold for treating Urological disorders such as urethral strictures and the like.

According to one embodiment of the present invention is a process of preparing autologous buccal epithelial cell suspension comprising harvesting the buccal mucosal tissue from subject, treating with chemical dissociation agent, washing with nutrient medium, separating the epidermis from dermis, preparing uniform epithelial cell suspension from epidermis by mincing and filtering, optionally seeding, harvesting the cellular monolayer with enzymes(s), centrifuging, discarding the supernatant, mixing with nutrient medium, analyzing the cell suspension, filling the cell suspension in V shaped 1 ml vials and optionally transporting to the same subject.

The subject is an adult human subject.

The autologous buccal epithelial cell suspension of the present invention is optionally mixed with gel while cystoscopically implanting the buccal epithelial cell suspension into the defect site of the urethra of the subject.

The defect may be urethral strictures.

The buccal mucosal tissue harvested from subject may be about 2.5×2.5 cm², preferably 2×1.5 cm², most preferred being 1×1.5 cm² of oral buccal mucosal tissue is harvested from subject.

The harvested buccal mucosal tissue maybe treated with chemical dissociation agent selected from trypsin, dispase, collagenase, trypsin-EDTA, pronase, hyaluronidase, elastase, papain and pancreatin. The amount of trypsin that used may be between 5 and 0.1% per volume of solution, preferably 2.5 to 0.25% most preferred being 0.5%. The time period for which the tissue sample is subjected to the trypsin solution may vary depending on the size of the buccal mucosal tissue, preferably for sufficient time to weaken the cohesive bonding between the tissue stratum, most preferred being 16 to 18 hours at 2-8° C.

Post dissociation the tissue sample is washed with nutrient medium selected from DMEM (Dulbecco's Modified Eagle's medium), EMEM(Eagle's Minimum Essential Medium), F12, IMDM (Iscove's Modified Dulbecco's Medium)and the like. Washing the tissue sample may involve either partial or complete immersion of the treated sample in the nutrient medium. Alternatively, and more preferably, the wash solution is dripped on the tissue sample in sufficient volume to remove and or significantly dilute any excess trypsin solution from the surface of the sample.

The nutrient medium used in the method should be capable or significantly reducing and more preferably removing the effect of the trypsin either by dilution or neutralization. The nutrient medium used in the method may preferably have the characteristics of being (i) capable of maintaining the viability of the cells until applied to a patient, and (ii) suitable for direct application to a region on a patient undergoing tissue grafting. The solution may be anything from a basic salt solution to a more complex nutrient solution. Preferably, the nutrient medium should contain various salts that resemble the substances found in body fluids; this type of solution is often called physiological saline. Phosphate or other non-toxic substances may also buffer the solution in order to maintain the pH at approximately physiological levels. A suitable nutrient medium that is particularly preferred is DMEM solution.

The tissue is subjected to separation of epidermis and dermis of the washed tissue and then minced, filtered to obtain epithelial cell suspension from epidermis followed by optional seeding in T-25 and/or T-75 and/or T-150 flask to enable cell multiplication. Cell multiplication is carried out for 2 weeks which is a one stage cell culture product (P0).

The cellular monolayer is harvested with enzymes selected from trypsin-EDTA, collagenase and the like, followed by centrifuging, discarding the supernatant and mixing the pellet with nutrient medium selected from IMDM, EMEM, DMEM and the like. The cell suspension is analyzed for Appearance, Sterility, Mycoplasma, Endotoxin, Cell Counting, Cell Viability, Cell Purity Test, Cell Characterization and Karyotyping Analysis and filled in V shaped 1 ml vials and optionally transported to the same subject at 2 to 8 degrees centigrade within 72 hours. Each 1 ml vial will comprise not less than 2.5 million cells (NLT 2.5 million cells) in 0.4 ml DMEM. 1 ml vial comprises 0.4 ml of autologous buccal epithelial cell suspension comprising 2.5 million cells. Typically, 2 vials comprising 0.4 ml of autologous buccal epithelial cell suspension each are used for strictureor defectof 4 cm size. Hence 0.2 ml (1.25 million cells) will be applied for 1 cm defect size.

Further, the autologous buccal epithelial cell suspension comprising analyzed and characterized cells may be cystoscopically implanted, optionally with gel, into the defect site of the urethra of the subject. The gel may be selected from biocompatible delivery system such as combination of fibrinogen and/or thrombin and/or thermo-reversible gelation polymer (TGP) gel and/or collagen and/or chitosan and the like.

According to another embodiment of the present invention is a method cystoscopically implanting the autologous buccal epithelial cell suspension into the defect site of the adult human urethra.

The method of cystoscopically implanting the autologous buccal epithelial cell suspension. into the defect site of the adult human urethra comprises preparation of the autologous buccal epithelial cell suspension, mixing with nutrient medium, analyzing the cell suspension, optionally filling and transporting the cell suspension in V shaped vials followed by cystoscopically implanting the autologous buccal epithelial cell suspension by optionally mixing with gel into the defect site of the adult human urethra.

The process of preparing autologous buccal epithelial cell suspension comprises harvesting the buccal mucosal tissue from subject, treating with chemical dissociation agent, washing with nutrient medium, separating the epidermis from dermis, preparing uniform epithelial cell suspension from epidermis by mincing and filtering, optionally seeding, harvesting the cellular monolayer with enzymes(s), centrifuging, discarding the supernatant, mixing with nutrient medium, analyzing the cell suspension, filling the cell suspension in V shaped 1 ml vials and optionally transporting to the same subject.

The buccal mucosal tissue harvested from subject may be about 2.5×2.5 cm², preferably 2×1.5 cm², most preferred being 1×1.5 cm² of oral buccal mucosal tissue is harvested from subject.

The harvested buccal mucosal tissue maybe treated with chemical dissociation agent selected from trypsin, dispase, collagenase, trypsin-EDTA, pronase, hyaluronidase, elastase, papain and pancreatin. The amount of trypsin that used may be between 5 and 0.1% per volume or solution, preferably 2.5 to 0.25% most preferred being 0.5%. The time period for which the tissue sample is subjected to the trypsin solution may vary depending on the size of the buccal mucosal tissue, preferably for sufficient time to weaken the cohesive bonding between the tissue stratum, most preferred being 16 to 18 hours at 2-8° C. Post dissociation the tissue sample is washed with nutrient medium selected from DMEM (Dulbeeco's Modified Eagle's medium), EMEM (Eagle's Minimum Essential Medium), F12, IMDM (Iscove's Modified Dulbecco's Medium) and the like. Washing the tissue sample may involve either partial or complete immersion of the treated sample in the nutrient solution. Alternatively, and more preferably, the wash solution is dripped on the tissue sample in sufficient volume to remove and or significantly dilute any excess trypsin solution from the surface of the sample.

The nutrient medium used in the method should be capable of significantly reducing and more preferably removing the effect of the trypsin either by dilution or neutralization. The nutrient medium used in the method may preferably have the characteristics of being (i) capable of maintaining the viability of the cells until applied to a patient, and (ii) suitable for direct application to a region on a patient undergoing tissue grafting. The medium may be anything from a basic salt solution to a more complex nutrient solution. Preferably, the nutrient medium should contain various salts that resemble the substances found in body fluids; this type of solution is often called physiological saline. Phosphate or other non-toxic substances may also buffer the solution in order to maintain the pH at approximately physiological levels. A suitable nutrient medium that is particularly preferred is DMEM solution.

The tissue is subjected to separation of epidermis and dermis of the washed tissue and then minced, filtered to obtain epithelial cell suspension from epidermis followed by optional seeding in T-25 and/or T-75 and/or T-150 flask and the like to enable cell multiplication. Cell multiplication is carried out for 2 weeks which is a one stage cell culture product (P0).

The cellular monolayer is harvested with enzymes selected from trypsin-EDTA, collagenase and the like, followed by centrifuging, discarding the supernatant and mixing the pellet with nutrient medium selected from IMDM, EMEM, DMEM and the like. The cell suspension is analyzed for Appearance, Sterility, Mycoplasma, Endotoxin, Cell Counting, Cell Viability, Cell Purity Test, Cell Characterization and Karyotyping Analysis and filled in V shaped 1 ml vials and optionally transported to the same subject at 2 to 8 degrees centigrade. The vial will comprise not less than 2.5 million cells (NLT 2.5 million cells) IN 0.4 ml DMEM. 1 ml vial comprises 0.4 ml of autologous buccal epithelial cell suspension comprising 2.5 million cells. Typically, 2 vials comprising 0.4 ml of autologous buccal epithelial cell suspension are used for strictureor defect of 4 cm size. Hence 0.2 ml (1.25 million cells) will be applied for 1 cm defect size.

The autologous buccal epithelial cell suspension of the present invention is optionally mixed with gel while cystoscopically implanting the autologous buccal epithelial cell suspension into the defect site of the urethra of the subject. The gel may be selected from biocompatible delivery system such as combination of fibrinogen and/or thrombin and/or thereto-reversible gelation polymer (TGP) gel and/or collagen and/or chitosan and the like.

The cystoscopic implantation may be typically carried out as follows
(1) Urethotomy is conducted to relieve the complete urethral stricture using an internal cystoscopic knife. Mainly at 12 O'clock position.
(2) Stricture area is visualized with scope.
(3) Foleys catheter of 14 French is passed through urethra and Foley catheter balloon is inflated.
(4) 6 French Urethroscope is passed through urethra and stricture area is visualized.
(5) Keeping 6 French scope in urethra, 5 French infant feeding tube is passed.
(6) Now, cystoscope and catheter along with infant feeding tube is present in urethra.
(7) With same cystoscopy, stricture area is visualized and tip of infant feeding tube is visualized and directed towards the stricture area at starting of the stricture at bladder side.
(8) Minimal traction of Foley catheter is given and saline flow is stopped.
(9) Stricture area to be dried out by suction of saline through infant feeding tube.
(10) Autologous buccal epithelial cell suspension is implanted at the stricture area with the help of biocompatible delivery system such as combination of fibrinogen and/or thrombin and/or thereto-reversible gelation polymer (TGP) gel and/or collagen and/or chitosan and the like through infant feeding tube at stricture area. Infant feeding tube is little withdrawn till another end of stricture.
(11) A method of final preparation of autologous buccal epithelial cell suspension for implantation at defect site of urethra comprising
   a. aspirating 1 ml of vial 1 contents, mixing with vial 2 contents and aspirating the contents into Syringe A;
   b. aspirating 1 ml vial 1 contents and mixing with vial 3 contents;
   c. drawing 0.2 ml from vial 3 and injecting into empty vial 4;
   d. drawing 0.4 ml+0.4 ml from vial(s) of autologous buccal epithelial cell suspension, injecting into vial 4 and aspirating the contents into Syringe B:
   e. placing Syringe A and B on the applicator/holder:
   f. fixing Y-shaped canula comprising a blunt needle to the two syringes; and
   g. implanting final 0.4 ml of final product into the detect site of the urethra of the subject by using cystoscopy.
(12) Biocompatible delivery system such as combination of fibrinogen and/or thrombin and/or thereto-reversible gelation polymer (TGP) gel and/or collagen and/or chitosan and the like is surrounded by Foleys catheter and covers stricture area in a cylindrical form.
(13) Cystoscope and infant tube is removed, and Foleys catheter is retained in urethra.
(14) Wait for 6 to 7 minutes holding penis in vertical position and maintain minimal traction on Foleys catheter.
(15) Penis is secured with strapping on abdomen, also Foleys catheter is strapped, so that Foleys catheter is firm and there is no movement.
(16) Urine bag is attached to Foleys catheter.

### DESCRIPTION OF THE DRAWINGS

FIG. 1—Autologous Buccal Epithelial Cell Culture Process.

FIG. 2—Process steps for the preparation of autologous buccal epithelial ceilsuspension.

FIG. 3A—Real time PCR based autologous buccal epithelial cell characterization (Amplification plot generated after real time PCR using GAPDH gene specific primers).

FIG. 3B—Real time PCR based autologous buccal epithelial cell characterization (Amplification plot generated after real time PCR using CK14+ gene specific primers).

FIG. 4A—Flow cytometry based autologous buccal epithelial cell characterization using CK14⁺ surface markers.

FIG. 4B—Flow cytometry based autologous buccal epithelial cell characterization test.

FIG. 5—Result of karyotyping Analysis performed at the autologous buccal epithelial cell manufacturing step.

FIG. 6—Schematic representation with duploject.

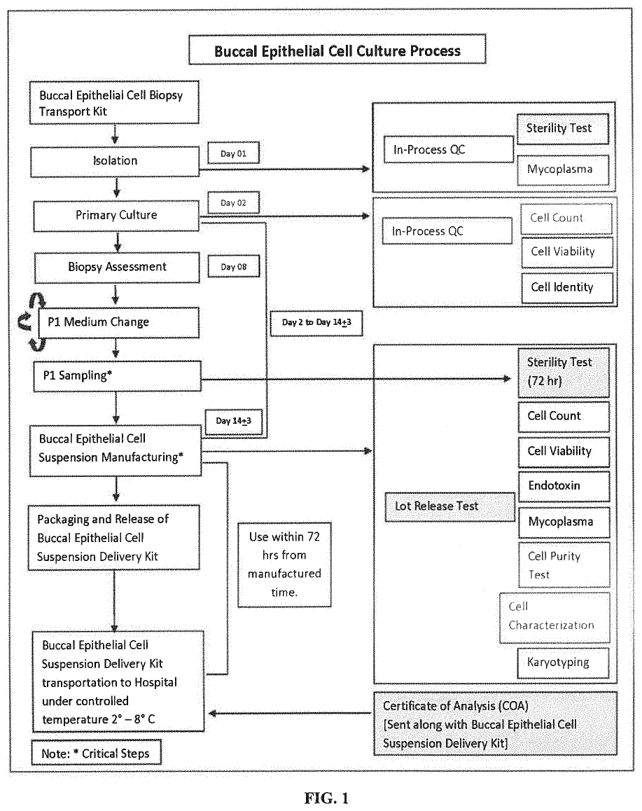

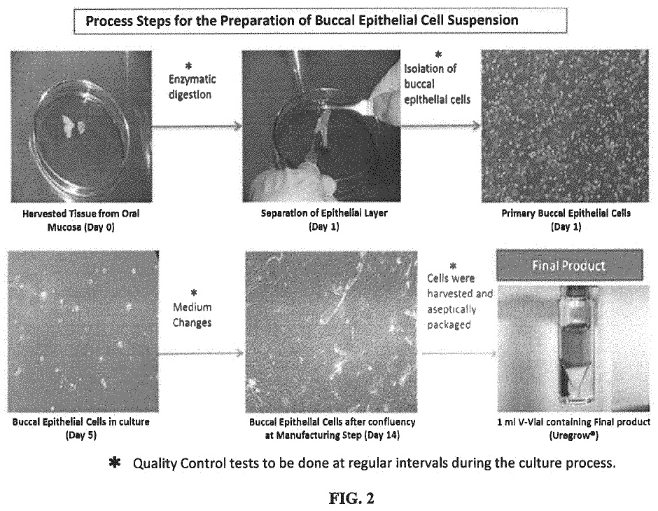

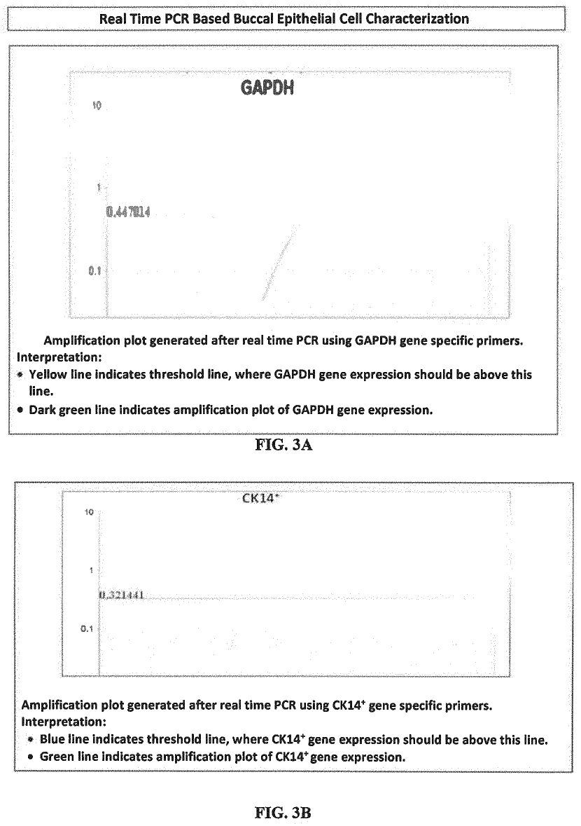

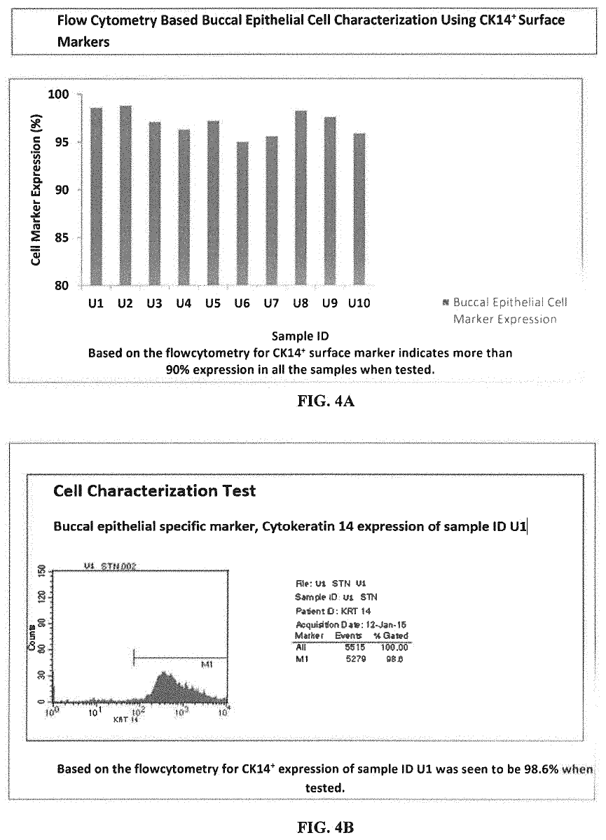

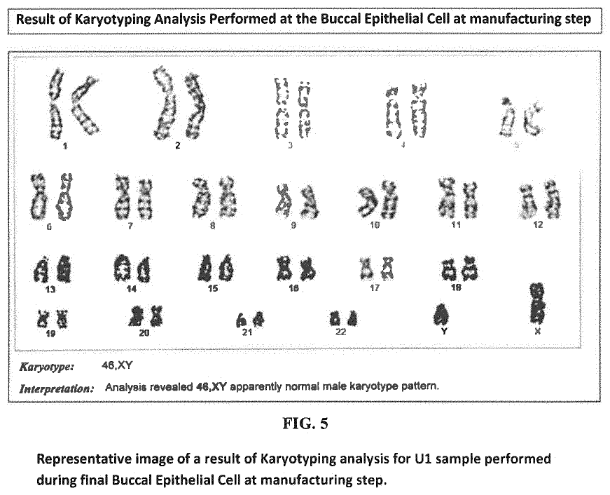

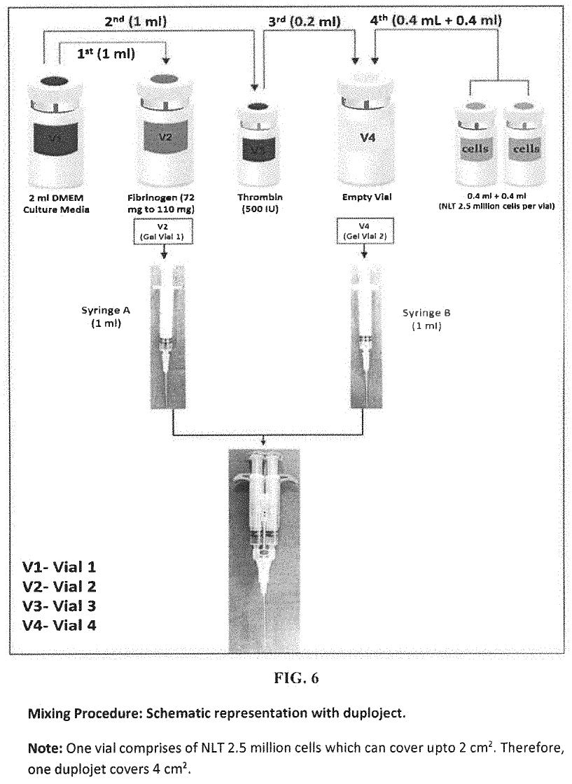

The following examples illustrate preferred embodiments in accordance with the present invention without limiting the scope of the invention.

### EXAMPLES

### Example 1

Oral mucosal tissue 1+1.5 cm² is harvested from the inner cheek region of an adult human patient with urethral stricture. The harvested tissue is placed in 0.5% enzyme trypsin in calcium and magnesium ion free phosphate buffer saline solution for 16 to 18 hours at 2-8° C. The tissue sample is removed from the solution and washed with DMEM solution. The cellular stratum of the tissue sample is separated with a forcep, minced and filtered to obtain uniform cell suspension of epithelial cells. The cells are suspended in DMEM medium and seeded with in T-25 flask.

The DMEM medium is replaced every alternate day and when the cell confluency is about 80 to 90% the cellular monolayer is harvested with enzyme trypsin-EDTA. The cellular suspension is subjected to centrifugation and supernatant is discarded. The pellet is mixed with nutrient medium. Appropriate number of cell suspension is filled in transparent V shaped 1 ml vial for transportation to the stricture site.

Quality control test(s) such as Appearance, Sterility, Mycoplasma, Endotoxin, Cell Counting, Cell Viability, Cell Purity Test, Cell Characterization and Karyotyping Analysis are conducted as mentioned below.

a) Appearance
Standard;
Red-Colored culture medium (DMEM), which contains mixed precipitated pale-white-colored autologous adult live cultured buccal epithelial cells. This content becomes turbid when shaken.

b) Sterility Test
Standard:
According to the Sterility Test of Indian Pharmacopoeia biological test methods section 2.2.11, if no evidence of microbial growth is found, the preparation under examination complies with the test for sterility.

c) Mycoplasma Test (PCR Method)
Standard:
It shall not detect any mycoplasma when tested according to the mentioned test.

d) Endotoxin Test
Standard:
According to biologics standard and Method D of Bacterial Endotoxins Testing Method under section 2.2.3 of the biological methods in Indian Pharmacopoeia 2007, there should be less than 3 EU/mL endotoxin during testing.

e) Cell Counting Test
Standard:
It shall be included ≥2,500,000 cells per 1 vial when tested.

f) Cell Viability Test
Standard:
Viable cells shall be over 80% of total cell count when tested.

g) Cell Purity Test
Standard:
Total albumin content shall be below 1.0 g/dL when tested.

h) Cell Characterization Test:
Standard:
Expression of positive stained cells for CD36⁺/CK5⁺/CK14⁺ antibodies shall be above 80% when tested.
Expression of negative stained cells for CD90⁻ antibodies shall be negligible when tested.

i) Karyotyping of Cells
Standard:
There should be no chromosomal abnormality on Karyogram imaging and analysis.

### Example 2: Analytical Data of Autologous Buccal Epithelial Cellsuspension From 10 Different Subjects

| | | | | | | | | Cell |
|---|---|---|---|---|---|---|---|---|
| | | | | | | | Cell | Characterization |
| | Tissue | Stricture | | Cell | Cell | | purity | (CK14⁺ |
| Sample | Size | Size | Cell | number | Viability | Endotoxin | test | expression) |
| No | (in cm) | (in cm) | Density | (×10⁶) | (%) | (EU/ml) | (g/dl) | by FACS |
| U1 | 1 × 1 | 2 | 0.4 ml | 4.90 | 96.27 | <0.769 | <0.204 | 98.60% |
| | | | (2.5 × 10\hat{ }6) | | | | | |
| U2 | 1.5 × 1 | 4 | 0.8 ml | 7.11 | 96.04 | <0.600 | <0.182 | 98.80% |
| | | | (5 × 10\hat{ }6) | | | | | |
| U3 | 1 × 1 | 1.5 | 0.3 ml | 5.60 | 94.67 | <0.930 | <0.205 | 97.10% |
| | | | (1.87 × 10\hat{ }6) | | | | | |
| U4 | 1 × 0.5 | 1 | 0.2 ml | 4.48 | 95.03 | <0.600 | <0.188 | 96.30% |
| | | | (1.25 × 10\hat{ }6) | | | | | |
| U5 | 1.5 × 1.5 | 4 | 0.8 ml | 8.17 | 95.96 | <0.600 | <0.189 | 97.20% |
| | | | (5 × 10\hat{ }6) | | | | | |
| U6 | 1.5 × 1 | 3 | 0.6 ml | 6.00 | 98.33 | <0.600 | <0.187 | 95.00% |
| | | | (3.75 × 10\hat{ }6) | | | | | |
| U7 | 1.5 × 1.5 | 4 | 0.8 ml | 8.31 | 97.62 | <0.600 | <0.188 | 95.60% |
| | | | (5 × 10\hat{ }6) | | | | | |
| U8 | 1.5 × 1 | 3 | 0.6 ml | 8.84 | 98.02 | <0.600 | <0.196 | 98.26% |
| | | | (3.75 × 10\hat{ }6) | | | | | |
| U9 | 1 × 1 | 2.5 | 0.5 ml | 7.91 | 98.02 | <0.600 | <0.208 | 97.60% |
| | | | (3.12 × 10\hat{ }6) | | | | | |
| U10 | 1.5 × 1.5 | 4 | 0.8 ml | 9.80 | 98.43 | <0.600 | <0.208 | 95.88% |
| | | | (5 × 10\hat{ }6) | | | | | |

Other Tests
Micro Sterility: negative in all samples
Mycoplasma: negative in all samples
Cell Characterization (CK14⁺ expression) by FACS & RT-PCR: demonstrating high levels of expression of CK14⁺ shows the high potency of buccal epithelial cells.
Cell Characterization (CD90⁻ expression) by RT-PCR: Negligible in all samples
Karyotypic Analysis: No chromosomal abnormalities in all samples

1. A process of preparing autologous buccal epithelial cell suspension comprising
(a) harvesting buccal mucosal tissue from subject;
(b) treating the tissue from (a) with chemical dissociation agent;
(c) washing the tissue sample from (b) with nutrient medium;
(d) separating the epidermis from dermis of the washed tissue;
(e) mincing, filtering to obtain uniform epithelial cell suspension from epidermis;
(f) optionally seeding to enable cell multiplication;
(g) harvesting the cellular monolayer with enzyme(s);
(h) centrifuging, discarding the supernatant;
(i) mixing with nutrient medium;
(j) analyzing the cell suspension;
(k) filling 0.4 ml of the cell suspension in V shaped 1 ml vials; and
(l) optionally transporting to the same subject as in (a).

2. A process of preparing autologous buccal epithelial cell suspension as claimed in claim 1 wherein the subject is an adult human subject.

3. A process of preparing autologous buccal epithelial cell suspension as claimed in claim 1 further comprising optionally mixing with gel while cystoscopically implanting the cell suspension into the defect site of the urethra of the subject; wherein the gel is selected from biocompatible delivery system such as combination of fibrinogen and/or thrombin and/or thermo-reversible gelation polymer (TGP) gel and/or collagen and/or chitosan and the like.

4. A process of preparing autologous buccal epithelial cell suspension as claimed in claim 1 wherein the chemical dissociation agent is selected from trypsin, dispase, collagenase, trypsin-EDTA, pronase, hyaluronidase, elastase, papain and pancreatin.

5. A process of preparing autologous buccal epithelial cell suspension as claimed in claim 1 wherein the nutrient medium is selected from DMEM, EMEM, F12, IMDM and the like.

6. A process of preparing autologous buccal epithelial cell suspension as claimed in claim 1 wherein the seeding is done in T-25 and/or T-75 and/or T-150 flask and the like.

7. A process of preparing autologous buccal epithelial cell suspension as claimed in claim 1 wherein the enzyme for harvesting is selected from trypsin-EDTA, collagenase and the like.

8. A process of preparing autologous buccal epithelial cell suspension as claimed in claim 1 wherein the analysis performed are Appearance, Sterility, Mycoplasma, Endotoxin, Cell Counting, Cell Viability, Cell Purity Test, Cell Characterization and Karyotyping Analysis.

9. A process of preparing autologous buccal epithelial cell suspension as claimed in claim 1 wherein the harvested buccal mucosal tissue is from the inner check of adult human and is about 1×1.5 cm².

10. A process of preparing autologous buccal epithelial cell suspension as claimed in claim 1 wherein the transportation is at 2 to 8 degree centigrade.

11. A method of cystoscopically implanting the autologous buccal epithelial cell suspension into the defect site of the adult human urethra comprising
(a) harvesting human buccal mucosal tissue from the inner cheek of the adult human;
(b) subjecting the tissue from (a) to chemical dissociation agent;
(c) washing the tissue sample from (b) with nutrient medium;
(d) separating the epidermis from dermis of the washed tissue;
(e) mincing, filtering to obtain uniform epithelial cell suspension from epidermis;
(f) optionally seeding to enable cell multiplication;
(g) harvesting the cellular monolayer with enzyme(s);
(h) centrifuging, discarding the supernatant;
(i) mixing with nutrient medium;
(j) analyzing the cell suspension;
(k) optionally filling and transporting the cell suspension in V shaped 1 ml vials; and
(l) optionally mixing with gel while cystoscopically implanting the autologous buccal epithelial cell suspension into the defect site of the adult human urethra.

12. A method of cystoscopically implanting the autologous buccal epithelial cell suspension into the defect site of the adult human urethra as claimed in claim 11 wherein the chemical dissociation agent is selected from trypsin, dispase, collagenase, trypsin-EDTA, pronase, hyaluronidase, elastase, papain and pancreatin.

13. A method of cystoscopically implanting the autologous buccal epithelial cell suspension into the defect site of the adult human urethra as claimed in claim 11 wherein the nutrient medium is selected from DMEM, EMEM, F12, IMDM and the like.

14. A method of cystoscopically implanting the autologous buccal epithelial cell suspension into the defect site of the adult human urethra as claimed in claim 11 wherein the seeding is done in T-25 and/or T-75 and/or T-150 flask and the like.

15. A method of cystoscopically implanting the autologous buccal epithelial cell suspension into the defect site of the adult human urethra as claimed in claim 11 wherein the enzyme is selected from trypsin-EDTA, collagenase and the like.

16. A method of cystoscopically implanting the autologous buccal epithelial cell suspension into the defect site of the adult human urethra as claimed in claim 11 wherein the analysis performed are Appearance, Sterility, Mycoplasma, Endotoxin, Cell Counting, Cell Viability, Cell Purity Test, Cell Characterization and Karyotyping Analysis.

17. A method of cystoscopically implanting the autologous buccal epithelial cell suspension into the defect site of the adult human urethra as claimed in claim 11 wherein the harvested human buccal mucosal tissue from the inner cheek of the human is about 1×1.5 cm².

18. A method of cystoscopically implanting the autologous buccal epithelial cell suspension into the defect site of the adult human urethra as claimed in claim 11 wherein the transportation is at 2 to 8 degree centigrade.

19. A method of cystoscopically implanting the autologous buccal epithelial cell suspension into the defect site of the adult human urethra as claimed in claim 11 wherein the gel is selected from biocompatible delivery system such as combination of fibrinogen and/or thrombin and/or theremo-reversible gelation polymer (TGP) gel and/or collagen and/or chitosan and the like.
a. Urethotomy is conducted to relieve the complete urethral stricture using an internal cystoscopic knife. Mainly at 12 O'clock position.
b. Stricture area is visualized with scope.
c. Foley's catheter of 14 French is passed through urethra and Foley catheter balloon is inflated.
d. 6 French Urethroscope is passed through urethra and stricture area is visualized.
e. Keeping 6 French scope in urethra, 5 French infant feeding tube is passed.
f. Now, cystoscope and catheter along with infant feeding tube is present in urethra.
g. With same cystoscopy, stricture area is visualized and tip of infant feeding tube is visualized and directed towards the stricture area at starting of the stricture at bladder side.
h. Minimal traction of Foley catheter is given and saline flow is stopped.
i. Stricture area to be dried out by suction of saline through infant feeding tube.
j. Autologous buccal epithelial cell suspension is implanted at the stricture area with the help of biocompatible delivery system such as combination of fibrinogen and/or thrombin and/or theremo-reversible gelation polymer (TGP) gel and/or collagen and/or chitosan and the like through infant feeding tube at stricture area. Infant feeding tube is little withdrawn till another end of stricture.
k. Biocompatible delivery system such as combination of fibrinogen and/or thrombin and/or theremo-reversible: gelation polymer (TGP) gel and/or collagen and/or chitosan and the like is surrounded by Foleys catheter and covers stricture area in a cylindrical form.
l. Cystoscope and infant tube is removed, and Foleys catheter is retained in urethra.
m. Wait for 6 to 7 minutes holding penis in vertical position and maintain minimal traction on Foleys catheter.
n. Penis is secured with strapping on abdomen, also Foleys catheter is strapped, so that Foleys catheter is firm and there is no movement.
o. Urine bag is attached to Foleys catheter.
